(19) United States
(12) Patent Application Publication
(10) Pub. No.: US 2022/0167784 A1
(43) Pub. Date: Jun. 2, 2022

(54) SANITIZATION UNIT, BEVERAGE PREPARATION MACHINE COMPRISING SUCH A SANITIZATION UNIT AND BEVERAGE PREPARATION METHOD IMPLEMENTING SUCH A BEVERAGE PREPARATION MACHINE

(71) Applicant: SOCIETE DES PRODUITS NESTLE S.A., Vevey (CH)

(72) Inventors: CHRISTIAN ABEND, Solon, OH (US); JEAN-MARC FLICK, Solon, OH (US); BERNARD KUNZLE, Lonay (CH)

(21) Appl. No.: 17/597,681

(22) PCT Filed: Jul. 20, 2020
(86) PCT No.: PCT/EP2020/070465

§ 371 Date: Jan. 18, 2022

(30) Foreign Application Priority Data
Jul. 19, 2019 (EP) 19187418.9

(51) Int. Cl. A47J 31/60; A47J 31/46

(52) U.S. Cl. CPC A47J 31/60; A47J 31/469; A47J 31/461

(57) ABSTRACT

The present invention relates to a sanitization unit (1) for sanitizing a liquid delivered to prepare a beverage. The sanitization unit (1) comprises an irradiation chamber (2) to receive liquid. The irradiation chamber (2) comprises: i) a liquid inlet (10) for introducing the liquid into the 5 irradiation chamber (2) and ii) a liquid outlet (12) for discharging the introduced liquid out of the irradiation chamber (2). An irradiation device (4) is configured to sanitize the liquid in the irradiation chamber (2). The liquid outlet (12) comprises a flow restrictor (12.1) to restrict a liquid flow when the pressure difference across the flow restrictor (12.1) is below a predetermined threshold, and to allow for a liquid flow with a higher flow rate when this pressure difference is equal to or greater than the predetermined threshold.

### TECHNICAL FIELD

The present invention relates to a sanitization unit for sanitizing a liquid to be delivered to a beverage preparation unit in order to prepare a beverage, for example by injecting the sanitized liquid into a product container. Further, the present invention relates to a beverage preparation machine comprising such a sanitization unit. Besides, the present invention relates to a beverage preparation method implementing such a sanitization unit.

The product container may enclose a nutritional composition or ingredients in a liquid, pasty or powdery form, such as an infant formula composition, milk-based ingredients or soya-based ingredients. With the present invention a ready-to-drink beverage may be prepared in a safe and hygienic manner. The present invention may be used to prepare a wide spectrum of beverages by using various product containers.

### BACKGROUND

Some of the currently available sanitization units comprise an irradiation chamber and an irradiation device intended to sanitize a liquid dispensed to prepare a beverage. The irradiation chamber has a liquid inlet and a liquid outlet so as to let the liquid flow in and, after irradiation, out of the irradiation chamber.

However, the present applicant has observed that the known sanitization units present some problems and drawbacks, which might arise from their designs, possibly from the arrangement of the liquid inlet and of the liquid outlet. In some instances, the liquid might not be sufficiently irradiated, hence insufficiently sanitized, in case its flow pattern transiently follows a short path in the irradiation chamber and/or makes the residence duration in the irradiation chamber too short.

### SUMMARY

In view of the above-mentioned problems and drawbacks, the present invention aims to improve the current implementations. An objective is to ensure an enhanced sanitization of the liquid and of the liquid receiving components before and/or during the beverage preparation, while enabling an optimal mechanical integration in a beverage preparation machine.

The objective is achieved by the embodiments defined in the independent claims. Advantageous implementations are further defined in the dependent claims.

An embodiment of the invention provides a sanitization unit, for sanitizing a liquid to be delivered to a beverage preparation unit in order to prepare a beverage, the sanitization unit comprising an irradiation chamber configured to receive an amount of liquid, the irradiation chamber comprising: i) a liquid inlet for introducing the liquid into the irradiation chamber and ii) a liquid outlet for discharging the introduced liquid out of the irradiation chamber, the sanitization unit further comprising an irradiation device configured to emit sanitizing radiations into the irradiation chamber so as to sanitize the liquid in the irradiation chamber. The irradiation device preferably comprising a UV light source. The liquid outlet comprises a flow restrictor configured to restrict a liquid flow via the liquid outlet to a restricted flow rate when the pressure difference across the flow restrictor is below a predetermined threshold value, and to allow for a liquid flow via the liquid outlet with a discharged flow rate higher than the restricted flow rate when the pressure difference across the flow restrictor is equal to or greater than the predetermined threshold value.

As the liquid begins to be introduced via the liquid inlet, the pressure in the irradiation chamber starts to increase. When the pressure is below the predetermined threshold value, the flow restrictor restricts the liquid flow via the liquid outlet to the restricted flow rate, for example to a null or negligible flow rate.

As the pressure builds up in the irradiation chamber, the introduced liquid may follow a long path inside the irradiation chamber, for example with a swirling motion, such that the introduced liquid may be irradiated by the irradiation device for a long enough period of time to ensure its proper sanitization.

Then, the flow restrictor allows for a liquid flow via the liquid outlet with a discharged flow rate that is higher than the restricted flow rate (e.g. null or negligible) when the pressure difference across the flow restrictor becomes equal and then greater than the predetermined threshold value. For example, if the restricted flow rate was null, then the sanitized liquid may begin to flow via the liquid outlet, hence out of the irradiation chamber. Downstream the liquid outlet the sanitized liquid may be injected into the beverage preparation unit, and possibly into a product container, to prepare the beverage in a safe and hygienic manner.

Thus, the sanitization unit may ensure an enhanced sanitization of the introduced liquid and of the liquid receiving components before and/or during the beverage preparation, while enabling an optimal mechanical integration in a beverage preparation machine.

In various implementations, the flow restrictor is configured such that the restricted flow rate is in a range from 0% to 80%, preferably from 20% to 80%, more preferably from 20% to 60%, of the liquid flow rate via the liquid inlet.

Thus, such a restricted flow rate may ensure that the pressure quickly builds up in the irradiation chamber, such that the liquid is sanitized intensively thanks to the formation of a turbulent rotating or swirling flow in the irradiation chamber.

In particular, the liquid flow rate via the liquid outlet and the liquid flow rate via the liquid inlet may be measured under the usual conditions for a beverage preparation, for example when the pressure in the liquid flowing via the liquid inlet ranges from 4 bar to 5 bar.

In various implementations, the flow restrictor may be configured such that the discharged flow rate is about 100% of the liquid flow rate via the liquid inlet.

Thus, a turbulent rotating or swirling fluidic motion is established in the irradiation chamber, which helps increasing the residence time of the liquid in the irradiation chamber and hence increase the irradiation dose received by the liquid. Also, the discharged flow rate may efficiently dissolve powder in the container. The discharged flow rate may present some fluctuations around the afore-mentioned value of 100% of the liquid flow rate via the liquid inlet depending for example on the configuration and service parameters.

In some implementations, a length of flow between the irradiation chamber and the flow restrictor may range from 0% to 100%, preferably from 0% to 50%, more preferably from 0% to 25% of the longest dimension of the irradiation chamber. Thus, the flow restrictor may be arranged close to or at the irradiation chamber, which limits to a negligible value the volume of liquid that is discharged via the liquid outlet before the pressure across the flow restrictor has reached the predetermined threshold value.

Preferably, the discharge line may be dimensioned as short (length) and as narrow (cross-sectional area) as possible to minimize microbial growth in the discharge line, by minimizing the inner surface available for microbial growth. The discharge line may represent the last fluidic portion extending between the flow restrictor and the beverage preparation unit.

In some implementations, the flow restrictor may be integrated in walls enclosing the irradiation chamber. Alternatively, the flow restrictor may be secured directly to the irradiation chamber. Thus, such configurations help minimize the volume of liquid discharged via the liquid outlet before the pressure across the flow restrictor has reached the predetermined threshold value.

In some implementations, the flow path between the irradiation chamber and the flow restrictor may extend along a straight line. Alternatively, the flow path between the irradiation chamber and the flow restrictor may extend along a curved line.

In various implementations, the flow restrictor may have a cross-sectional area ranging from 0.28 mm² to 0.79 mm², preferably from 0.28 mm² to 0.50 mm², more preferably from 0.35 mm² to 0.43 mm², still more preferably equal to about 0.38 mm².

Thus, such a cross-sectional area in the flow restrictor may appropriately restrict the liquid flow discharged via the liquid outlet. Indeed, such a cross-sectional area may strike a balance between i) a flow restrictor that would insufficiently restrict the liquid flow and hence let an insufficiently sanitized liquid flow toward the beverage preparation unit, and ii) a flow restrictor that would excessively restrict the liquid flow and hence too much reduce the liquid velocity into the beverage preparation unit, and eventually into the product container. indeed, a high enough liquid velocity may be required in order to form a water jet ensuring a proper dissolution of the product in the container.

In some implementations, the irradiation device may comprise a UV light source, preferably at least one UV LED, more preferably several UV LEDs arranged for example as an array of UV LEDs. The at least one UV LED may be comprised of a solid state electroluminescent diode configured to emit UV light. The LEDs may be selected to provide a monodispersed light spectrum in the UVB-UVC spectrum (255-300 nm), for example a spectrum centered at 265 nm or 280 nm. Thus, the UV LEDs may be compactly arranged and hence simplify their integration into a beverage preparation machine.

In some implementations, the irradiation device may be configured to emit sanitizing radiations having a fluence of about 20 to 80 mW/cm² and a fluence rate of at least 40 to 90 mi/cm². Thus, the irradiation device provides a fairly high degree of sanitization, typically of about Log 4 to Log 5 for MS2 phages (Virus surrogate) and a Log 5 to Log 6 for the majority of bacteria.

When the liquid is pumped through the sanitization unit it is exposed to the sanitizing radiations into the irradiation chamber. The degree of sanitization depends on 1) the fluence or the power of emitted sanitizing radiations (mW/cm²) and 2) the time of exposure or dose or fluence rate (mJ/cm²). The higher the fluence rate or the fluence, the higher the degree of sanitization of the introduced liquid.

Thus, the irradiation device may appropriately irradiate the irradiation chamber and the introduced liquid received therein.

In some implementations, the irradiation chamber may substantially have a spheroidal shape, preferably a spherical shape. Such shape makes it possible for the irradiation device to reach most or all of the surface and volume of the irradiation chamber, directly and/or by reflection on walls defining the irradiation chamber. Thus, the irradiation chamber and the introduced liquid received therein may be intensively sanitized.

In some implementations, the irradiation device may define a lateral surface of the irradiation chamber. For example, the surface defined by the irradiation device may extend vertically when the sanitization unit is in its service configuration. In some implementations, the surface of the irradiation device may be substantially planar.

In some implementations, the walls defining the irradiation chamber may be made of a material reflecting UV radiations, for example of a material including or constituted by polytetrafluoroethylene (PTFE).

In various implementations, the flow restrictor may comprise a non-return valve, the non-return valve being preferably configured to open only in a direction downstream with respect to the irradiation chamber.

Thus, the flow restrictor may be separated from and secured to the irradiation chamber. For example, the non-return valve may be integrated into a beverage preparation unit that lies next to or distant from the sanitization unit.

In some implementations, the flow restrictor may have an adjustable cross-section. For example, the sanitization unit may further comprise an actuator configured to adjust or vary the adjustable cross-section depending on a control signal.

In various implementations, the predetermined threshold value may range from 2 bar to 8 bar, preferably from 4 bar to 5 bar.

Thus, the predetermined threshold value makes it possible for the pressure to build up rapidly in the irradiation chamber, hence to sanitize the introduced liquid intensively.

In various implementations, the liquid outlet may be located in a lowermost region of the irradiation chamber, more preferably close to or at the lowest level of the irradiation chamber.

Thus, the gravity may help the discharged liquid to flow via the liquid outlet.

In the present disclosure, the terms “uppermost”, “lowermost”, “upper”, “lower”, “above” and the like refer to the service configuration of the sanitization unit, hence when it is assembled in a beverage preparation machine and the liquid flows therethrough in order to prepare a beverage. In the present disclosure, the terms “upstream” and “downstream” refer to the direction of flow of a liquid during the preparation of a beverage. For example, the liquid supply is located upstream the sanitization unit.

In various implementations, the liquid inlet may be located in a lateral side region of the irradiation chamber, preferably distant from the liquid outlet, more preferably substantially opposite the liquid outlet, the liquid inlet being preferably oriented toward the irradiation device.

In various implementations, the sanitization unit may further comprise an air inlet for letting air enter the irradiation chamber and evacuate the liquid out of the irradiation chamber via the liquid outlet, the air inlet being preferably located in an uppermost region of the irradiation chamber and more preferably close to or at the highest level of the irradiation chamber.

Thus, the air entered via the air inlet may help empty the irradiation chamber. Further, the irradiation chamber may be drained and dried out by the entered air and then sanitized by the irradiation device after preparation of a beverage, hence before preparing the next beverage.

In various implementations, the sanitization unit may further comprise a non-return valve fluidly connected to the air inlet, wherein the non-return valve is preferably fitted in a wall defining the irradiation chamber, the non-return valve being preferably arranged close to or at the irradiation chamber such that most or all of wettable surfaces of the non-return valve are partially or totally exposed to the radiations of the irradiating device.

In some implementations, the irradiation device may be positioned with respect to the irradiation chamber so as to be distant from the air inlet, the irradiation device being preferably positioned opposite the air inlet, the irradiation device being preferably located in the lateral side region of the irradiation chamber and/or next to the liquid inlet.

Thus, the air inlet and the wettable surfaces of the non-return valve may be appropriately disinfected by the irradiation device.

In various implementations, the irradiation device may be positioned with respect to the irradiation chamber so as to be distant from the liquid outlet, the irradiation device being preferably positioned opposite the liquid outlet, the irradiation device being preferably located in the lateral side region of the irradiation chamber and/or next to the liquid inlet.

Thus, the wettable surfaces of the liquid outlet and of the air inlet may be sanitized by the irradiating device. These wettable surfaces are the surfaces that may be in contact with the introduced liquid, as they form a dead volume. Preferably, the wettable surfaces of the liquid outlet and of the air non-return valve are oriented toward the irradiating device.

In some implementations, the irradiation device may comprise a protective window for letting the emitted sanitizing radiations pass through, the protective window being and arranged to fluidly separate the irradiation chamber from the rest of the irradiation device. Thus, the protective window may protect the irradiating device while letting its radiations through to irradiate the irradiation chamber. In some implementations, the protective window may be made of quartz.

In some implementations, the irradiation device may comprise a sealing member arranged to seal the periphery of the protective window with respect to the irradiation chamber. Preferably, the sealing member may be arranged flush with the wall defining the irradiation chamber close to or at the protective window. Thus, no dead volume is formed around the protective window, which avoids the growth of microorganisms.

In some implementations, the irradiation chamber may be defined by at least two parts assembled together so as to substantially enclose the irradiation chamber, the sanitization unit further comprising sealing elements arranged between the at least two parts, the sealing elements being arranged flush with the walls defining the irradiation chamber close to or at the sealing elements, the sealing elements being arranged to be at least partially exposed to the radiations. Thus, no dead volume is formed around the sealing elements, which avoids the growth of microorganisms.

In some implementations, the liquid inlet may be configured to introduce the liquid substantially tangentially to a surface of the irradiation chamber, preferably tangentially to a surface of the lateral side region of the irradiation chamber, or tangentially to a surface of the irradiation device.

As the liquid is introduced tangentially to a surface of the irradiation chamber, the liquid may promote a swirling flow inside the irradiation chamber, in particular when the irradiation chamber is full of liquid. Thus, the introduced liquid received in the irradiation chamber may be sanitized during an appropriate residence duration.

Another embodiment of the present invention provides a beverage preparation machine comprising:
a sanitization unit, for sanitizing a liquid to be delivered to a beverage preparation unit in order to prepare a beverage, the sanitization unit comprising an irradiation chamber configured to receive an amount of liquid, the irradiation chamber comprising: i) a liquid inlet for introducing the liquid into the irradiation chamber and ii) a liquid outlet for discharging the introduced liquid out of the irradiation chamber, the sanitization unit further comprising an irradiation device configured to emit sanitizing radiations into the irradiation chamber so as to sanitize the liquid in the irradiation chamber,
a beverage preparation unit fluidly connected to the liquid outlet for preparing a beverage with the sanitized liquid,
a liquid supply unit fluidly connected to the liquid inlet and configured to supply a liquid to the beverage preparation unit via the liquid inlet, the irradiation chamber and the liquid outlet, and
a flow restrictor arranged between the irradiation chamber and the beverage preparation unit, the flow restrictor being configured to restrict a liquid flow via the liquid outlet to a restricted flow rate when the pressure difference across the flow restrictor is below a predetermined threshold value, and to allow for a liquid flow via the liquid outlet with a discharged flow rate higher than the restricted flow rate when the pressure difference across the flow restrictor is equal to or greater than the predetermined threshold value.

Thus, the beverage preparation machine may ensure an enhanced sanitization of the liquid and of the liquid receiving components before and/or during the beverage preparation, while enabling an optimal mechanical integration of the sanitization unit.

In some implementations, the beverage preparation machine may further comprise at least one of:
a liquid pump configured to displace the liquid toward the beverage preparation unit via the sanitization unit,
a heating system arranged between the liquid supply and the sanitization unit, the heating system being configured to heat up the liquid during preparation of a beverage,
an air pump configured to displace air toward the air inlet, and
a machine control unit configured to control at least one of the liquid supply unit, the air supply, the beverage preparation unit, and the sanitization unit.

In some implementations, the beverage preparation machine may further comprise an end valve, preferably a check valve, arranged in a discharge line fluidly connecting the liquid outlet and the beverage preparation unit, the end valve being configured to open only in one direction from the liquid outlet to the beverage preparation unit and only when the pressure difference across the end valve exceeds a given pressure difference threshold.

In various implementations, the sanitization unit may be located above at least one of the beverage preparation unit and the liquid supply unit, the sanitization unit being preferably located in an uppermost position of the beverage preparation machine.

As the sanitization unit lies above the beverage preparation unit, gravity may enhance the emptying of the irradiation chamber and the drainage of the liquid out of the irradiation chamber toward the discharge line and the beverage preparation unit.

In some implementations, the sanitization unit may be located on a head of the beverage preparation machine.

In various implementations, the beverage preparation machine may further comprise a discharge line fluidly connecting the liquid outlet and the beverage preparation unit, the discharge line being preferably dimensioned in accordance with a first requirement for minimizing microbial growth in the discharge line and a second requirement for minimizing a pressure drop across the discharge line.

The dimensions of the discharge line are selected as a compromise between the requirement to minimize the microbial growth therein and the requirement for the pump to deliver a suitable liquid flow rate with a minimum pressure drop across the pump.

In various implementations, the beverage preparation machine may further comprise a heating device arranged to transfer heat to at least a portion of the discharge line fluidly connecting the liquid outlet and the beverage preparation unit, the discharge line being preferably made of a thermally conductive material.

Thus, the heating device may efficiently dry and sanitize the discharge line after the beverage preparation in order to avoid the microbial growth therein.

Thus, the discharge line may be sanitized by heat, for example before and/or after preparation of a beverage. In case the discharge line has a fairly high thermal conductivity, the heat transferred locally by the heating device may spread to the whole discharge line, thus achieving a complete sanitization thereof.

In some implementations, the discharge line may be made of a metallic tube, for example made of stainless steel. In some implementations, the heating device may comprise:
a heating element arranged close to or around the discharge line, the heating element preferably having the shape of a cartridge, a torus, a cylinder or a helix,
a temperature sensor arranged to measure the temperature of the heating element or a portion of the discharge line, temperature sensor optionally being a thermal measuring resistance of the NTC type or a thermocouple and
a heat control unit configured to control the temperature of the heating element so as to heat the liquid present in the discharge line up to a temperature ranging from 65° C. to 90° C.

A further embodiment of the present invention provides a method for preparing a beverage, the method comprising:
implementing a beverage preparation machine as afore-described,
activating the liquid supply unit to deliver a liquid via the liquid inlet to the irradiation chamber, wherein the flow restrictor restricts a liquid flow via the liquid outlet to the restricted flow rate when the pressure difference across the flow restrictor is below the predetermined threshold value,
activating the irradiation device at least during the delivery of liquid to the irradiation chamber in order to sanitize the delivered liquid,
keeping the liquid supply unit running, wherein the flow restrictor allows for a liquid flow, via the liquid outlet and to the beverage preparation unit, with the discharged flow rate when the pressure difference across the flow restrictor is equal to or greater than the predetermined threshold value,
optionally, increasing the liquid flow supplied to the irradiation chamber via the liquid inlet after the pressure difference across the flow restrictor has become equal to or greater than the predetermined threshold value,
optionally, regulating the liquid flow supplied to the irradiation chamber via the liquid inlet so as to allow for the liquid to remain in the irradiation chamber for a predefined residence time before being discharged out of the irradiation chamber via the liquid outlet, and
preparing a beverage in the beverage preparation unit with the sanitized liquid.

Thus, the beverage preparation method may ensure an enhanced sanitization of the liquid and of the liquid receiving components before and/or during the beverage preparation.

It has to be noted that all devices, elements, components, members, units and means described in the present application could be implemented in any technically applicable combination of the implementation forms. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in any technically applicable combination of the implementation forms.

### BRIEF DESCRIPTION OF DRAWINGS

The above described aspects and implementation forms of the present invention will be explained in the following description of specific embodiments and aspects in relation to the enclosed drawings, in which:

FIG. 1 is a schematic perspective view illustrating a sanitization unit according to an embodiment of the present invention;

FIG. 2 is a schematic perspective view, at an angle different from FIG. 1, illustrating the sanitization unit of FIG. 1;

FIG. 3 is a schematic perspective view, with a cross-section along plane III on FIG. 2, illustrating the sanitization unit of FIG. 1;

FIG. 4 is a schematic hydraulic diagram illustrating a beverage preparation machine according to an embodiment of the present invention and including the sanitization unit of FIG. 1;

FIG. 5 is a schematic hydraulic diagram illustrating the beverage preparation machine of FIG. 4 during a beverage preparation;

FIG. 6 is a schematic hydraulic diagram illustrating the beverage preparation machine of FIG. 4 after a beverage preparation and during a drainage process of the beverage preparation machine;

FIG. 7 is a schematic perspective view illustrating a part of the beverage preparation machine of FIG. 4 including the sanitization unit of FIG. 1;

FIG. 8 is a schematic perspective view, at an angle different from FIG. 7, illustrating a part of the beverage preparation machine of FIG. 4 including the sanitization unit of FIG. 1;

FIG. 9 is a schematic perspective view, at an angle different from FIGS. 7 and 8, illustrating the part of the beverage preparation machine of FIG. 7;

FIG. 10 is a schematic perspective view, at an angle different from FIGS. 7 to 9, illustrating the part of the beverage preparation machine of FIG. 7;

FIG. 11 is a schematic flowchart illustrating a beverage preparation method according to an embodiment of the present invention;

FIG. 12 is a schematic flowchart illustrating in detail a drainage process performed in the beverage preparation method of FIG. 11.

### DETAILED DESCRIPTION

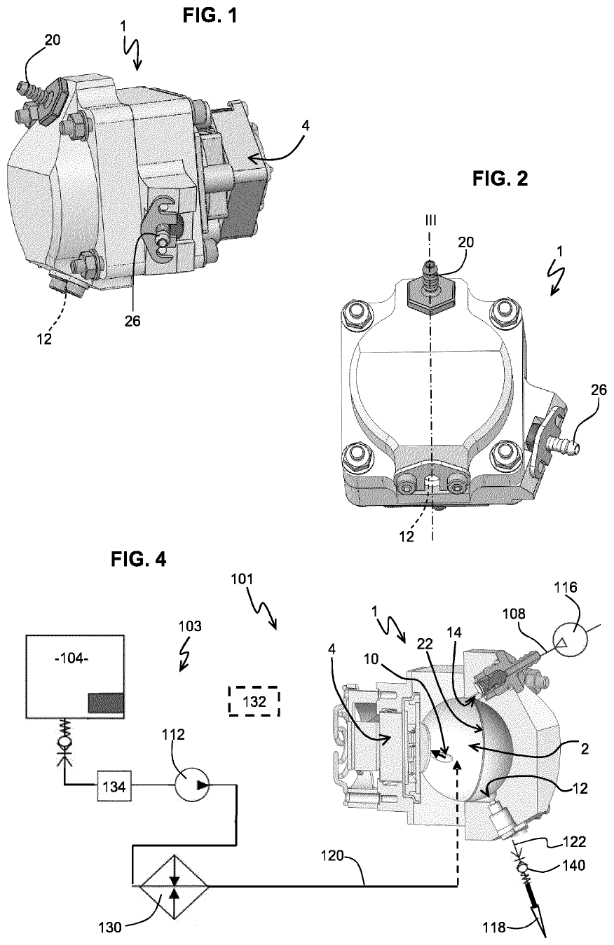

FIGS. 1 to 3 illustrate a sanitization unit 1 for sanitizing a liquid to be delivered to a beverage preparation unit 102, visible in FIGS. 4 to 6 in order to prepare a beverage. For example, the sanitization unit 1 may be used to prepare an infant formula preparation when the beverage preparation unit 102 receives a not shown product container enclosing an infant formula composition. In FIGS. 1 to 3, the sanitization unit 1 is represented in the service configuration.

The sanitization unit 1 comprises an irradiation chamber 2 configured to receive liquid. The irradiation chamber 2 comprises a liquid inlet 10 for introducing the liquid into the irradiation chamber 2. The irradiation chamber 2 comprises a liquid outlet 12 for discharging the introduced liquid out of the irradiation chamber 2.

The liquid inlet 10 is located in a lateral side region 2.3 of the irradiation chamber 2, in particular substantially at the middle height of the irradiation chamber 2. The liquid inlet 10 may be distant from, preferably opposite, the liquid outlet 12. The liquid inlet 10 may be oriented toward the irradiation device 4, so as to be irradiated and sanitized by the irradiation device 4.

The liquid outlet 12 is located in a lowermost region 2.1 of the irradiation chamber 2. The liquid outlet 12 may be located close to or at the lowest level of the irradiation chamber 2. The liquid outlet 12 and the liquid inlet 10 may be substantially oppositely arranged. The introduced liquid may follow a long path inside the irradiation chamber 2, for example with a swirling motion, such that the introduced liquid may be irradiated for a long enough period of time to ensure its proper sanitization.

The sanitization unit 1 further comprises an irradiation device 4 configured to emit sanitizing radiations into the irradiation chamber 2. The irradiation device 4 may be located in the lateral side region 2.3 of the irradiation chamber 2 and next to the liquid inlet 10. The irradiation device 4 may be arranged distant from the liquid outlet 12, preferably substantially opposite the liquid outlet 12.

The irradiation device 4 may be configured to emit sanitizing radiations having a fluence of about 20 to 80 mW/cm² and a fluence rate of at least 40 to 90 mi/cm², thus enabling a fairly high degree of sanitization.

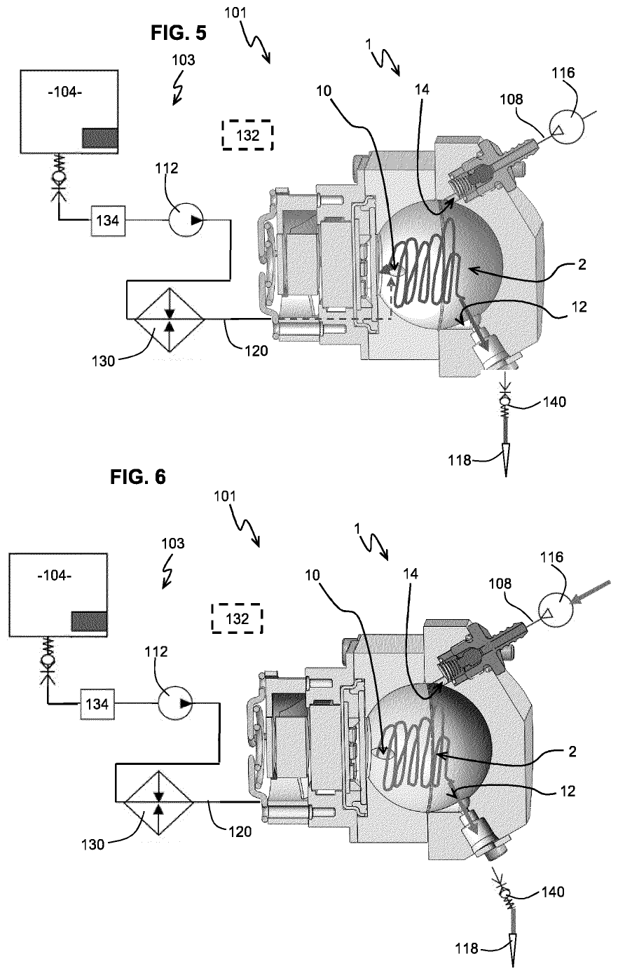

As visible in FIG. 6 the irradiation device 4 may include a UV light source comprising an array of four UV LEDs 6, two of which are visible in FIG. 5. The UV LEDs may be comprised of solid state electroluminescent diodes and selected to provide a monodispersed light spectrum in the UVB-UVC spectrum (255-300 nm). The UV LEDs 6 may be powered by a not shown DC power source providing a voltage of between 9 V and 12 V and a current of at least 1.2 A. The power consumption of the irradiation device 4 may range from 5 W to 13 W.

The sanitization unit 1 may further include a not shown UV sensor configured to provide a measure representative of the actual radiations, e.g. of the fluence, of the UV LEDs 6 in order to ensure that the UV LEDs 6 keep operating properly.

The liquid outlet 12 comprises a flow restrictor 12.1, which is configured to:
restrict a liquid flow via the liquid outlet 12 to a restricted flow rate when the pressure difference across the flow restrictor 12.1 is below a predetermined threshold value, and
to allow for a liquid flow via the liquid outlet 12 with a discharged flow rate higher than the restricted flow rate when the pressure difference across the flow restrictor 12.1 is equal to or greater than the predetermined threshold value.

In particular, the predetermined threshold value may be selected to be about 4 bar.

In particular, the flow restrictor may be configured such that the restricted flow rate, hence via the liquid outlet 12, may range from 0% to 80%, and be for example 20%, of the liquid flow rate via the liquid inlet 10, when the pressure difference across the flow restrictor 12.1 is below the predetermined threshold value. Besides, the flow restrictor 12.1 may be configured such that the discharged flow rate is about 100% of the liquid flow rate via the liquid inlet 10.

A length of flow between the irradiation chamber 2 and the flow restrictor 12.1 may range from 0% to 100%, for example be about 5%, of the longest dimension L2 of the irradiation chamber 2. The flow restrictor 12.1 may be arranged close to or at the irradiation chamber 2. The flow restrictor 12.1 may extend along a straight line.

The flow restrictor 12.1 may have a cross-sectional area of about A1% of the cross-sectional area of the liquid inlet. The flow restrictor 12.1 may have a cross-sectional area of about 0.38 mm².

In the example of FIGS. 1 to 4, the flow restrictor 12.1 is integrated in walls enclosing the irradiation chamber 2. The flow restrictor 12.1 may be formed by at least one restricted cross-section 12.2. Besides, in the example of FIGS. 1 to 4, the walls enclosing the irradiation chamber 2 may have bores 12.2 and 12.3, for example threaded bores, which are configured for fastening a not shown connector or non-return valve.

The irradiation chamber 2 may further comprise an air inlet 14 for letting air enter the irradiation chamber 2 and evacuate the liquid out of the irradiation chamber 2 via the liquid outlet 12, in particular after preparation of a beverage (FIG. 5). The air inlet 14 may be located in the uppermost region 2.2, preferably close to or at the highest level, of the irradiation chamber 2. The air inlet 14 may be located in the highest level of the irradiation chamber 2 as in FIGS. 1 to 4. The irradiation device 4 may be arranged distant from the air inlet 14, preferably substantially opposite the air inlet 14.

The sanitization unit 1 may further comprise a non-return valve 20, which is fluidly connected to the air inlet 14. The non-return valve 20 may be fitted in a wall defining the irradiation chamber 2. The non-return valve 20 may be arranged close to or at the irradiation chamber 2 such that most or all of wettable surfaces of the non-return valve 20 may be partially or totally exposed to the radiations of the irradiating device 40. The arrangement of the air non-return valve 20 opposite the irradiation device 4 enhances the sanitization of the wettable surfaces of the air non-return valve 20.

In the example of FIGS. 1 to 4, the irradiation chamber 2 may have three ports including the liquid inlet 10, the air inlet 14 and the liquid outlet 12. Each of these three ports may emerge in or be directly connected the irradiation chamber 2.

The liquid inlet 10 may be configured to introduce the liquid substantially tangentially to a surface of the irradiation chamber 2, preferably of the lowermost region 2.1. The liquid may promote a swirling flow inside the irradiation chamber 2, such that the liquid may be sanitized during an appropriate residence duration.

The irradiation device 4 may comprise a protective window 16 for letting the emitted sanitizing radiations pass through it. The protective window 16 may be arranged to separate the irradiation chamber 2 from the rest of the irradiation device 4.

The irradiation device 4 may further comprise a sealing member 18 arranged to seal the periphery of the protective window 16 with respect to the irradiation chamber 2. The protective window 16 and the sealing member 18 may be integrally assembled with the irradiation device 4.

The protective window 16 may be arranged to fluidly separate the irradiation device 4, in particular the UV LEDs 6, from the introduced liquid received in the irradiation chamber 2. The protective window 16 may be made of quartz.

The sealing member 18 may be arranged to seal the protective window 16 with respect to the introduced liquid received in the irradiation chamber 2. The sealing member 18 may be arranged flush with the wall defining the irradiation chamber 2 close to or at the protective window 16. Thus, no dead volume is formed around the protective window 16, which avoids the growth of microorganisms.

The shape of the irradiation chamber 2 is designed to make it possible for the irradiation device 4 to reach most or all of the surface and volume of the irradiation chamber 2. Thus, the irradiation chamber and the introduced liquid received therein may be appropriately sanitized.

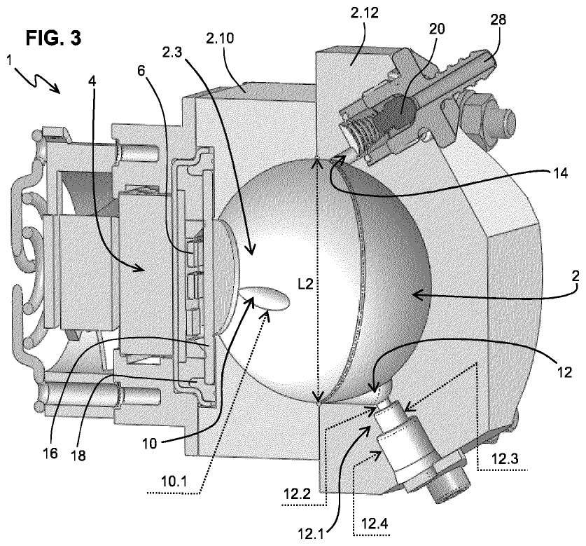

The irradiation chamber 2 may substantially have a spheroidal shape. The surfaces defining the irradiation chamber 2 of FIGS. 1 to 4 may form a spherical shape, except the upper surface 4.1 of the irradiation device 4. Indeed, the upper surface 4.1 may be substantially planar and extend substantially horizontally when the sanitization unit 2 is in its service configuration (FIGS. 3 and 4). The upper surface 4.1 may be defined by the protective window 16.

The UV LEDs 6 may be configured to emit sanitizing radiations under a solid angle covering most or all of the surfaces forming the irradiation chamber 2, which surfaces define the spherical shape of the irradiation chamber 2 in FIGS. 1 to 4.

Further, the walls defining the irradiation chamber 2 may be made of a material reflecting UV radiations, for example of a material including polytetrafluoroethylene (PTFE). The reflection of the UV radiations can enhance the sanitization of the volume and surfaces of the irradiation chamber 2. In particular, the reflected UV radiations may reach portions of the irradiation chamber 2 that are not directly irradiated by the UV radiations emitted by the UV LEDs 6.

The irradiation device 4 may define a lateral surface of the irradiation chamber 2. This lateral surface defined by the irradiation device may extend vertically when the sanitization unit 1 is in its service configuration. This lateral surface of the irradiation device 4 may be substantially planar.

The irradiation device 4 may include a PCB (Printed circuit board) 4.2, on which the UV LEDs 6 may be arranged. The PCB 4.2 may be formed of an aluminum or aluminum alloy substrate. The PCB 4.2 may integrate the not shown UV sensor. The irradiation device 4 may further include a heat sink 4.4, on which the PCB 4.2 may be mounted in order to evacuate the heat generated by the UV LEDs 6. Cooling the UV LEDs 6 avoids degrading their performances as well as a shift in their light spectrum, hence obtain a reliable sanitization performance.

Also, the liquid entering in the sanitization unit 1 may serve as a coolant in order to evacuate part or all of the heat generated by the UV LEDs 6. This evacuated heat does not much influence the temperature of the liquid since the liquid volume flowing through the sanitization unit 1 is quite large.

The PCB 4.2 may include a NTC temperature sensor configured to deliver a signal representative of the temperature of the UV LEDs 6. This signal may be used to manage a safety check as the temperature of the UV LEDs 6 may be monitored. Preferably, the temperature of the UV LEDs 6 should not exceed 55° C.

The irradiation chamber 2 may be defined by at least two parts, for example a first part 2.10 and a second part 2.12. In the example of FIGS. 1 to 4, the first part 2.10 accommodates the liquid inlet 10, while the second part 2.12 accommodates the liquid outlet 12. The first part 2.10 and the second part 2.12 may be assembled together so as to substantially enclose the irradiation chamber 2.

The sanitization unit 1 may further comprise a sealing element 22, which is arranged between the first part 2.10 and the second part 2.12. The sealing element 22 may be arranged flush with the walls defining the irradiation chamber 2 close to or at the sealing element 22. Besides, the sealing element 22 may be arranged to be at least partially exposed to the UV radiations emitted by the UV LEDs 6. Thus, no dead volume is formed around the sealing element 22, which prevents the growth of microorganisms.

As illustrated in FIGS. 3 and 4, the sanitization unit 1 may also comprise a not shown liquid inlet connector, a not shown liquid outlet connector and an air inlet connector 20. Respective channels may extend through the first part 2.10 and the second part 2.12 so as to fluidly connect i) the liquid inlet connector to the liquid inlet 10, ii) the liquid outlet connector to the liquid outlet 12, and iii) the air inlet connector 20 to the air inlet 14.

FIGS. 4 to 6 illustrate a beverage preparation machine 101 for preparing a beverage by delivering an amount of liquid to the beverage preparation unit 102 and from there to a not shown product container.

The beverage preparation machine 101 comprises:
the sanitization unit 1,
the beverage preparation unit 102, which is fluidly connected to the liquid outlet 12 for preparing a beverage with the sanitized liquid,
a liquid supply unit 103, which is fluidly connected to the liquid inlet 10 and configured to supply a liquid to the beverage preparation unit 102 via i) the liquid inlet 10, ii) the irradiation chamber 2 and iii) the liquid outlet 12, and
the flow restrictor 12.1, which is arranged between the irradiation chamber 2 and the beverage preparation unit 102.

The beverage preparation machine 101 may further comprise a discharge line 122 fluidly connecting the liquid outlet 12 and the beverage preparation unit 102. The discharge line 122 may be dimensioned in accordance with a first requirement for minimizing microbial growth in the discharge line 122 and a second requirement for minimizing a pressure drop across the discharge line 122, hence across the liquid pump 112.

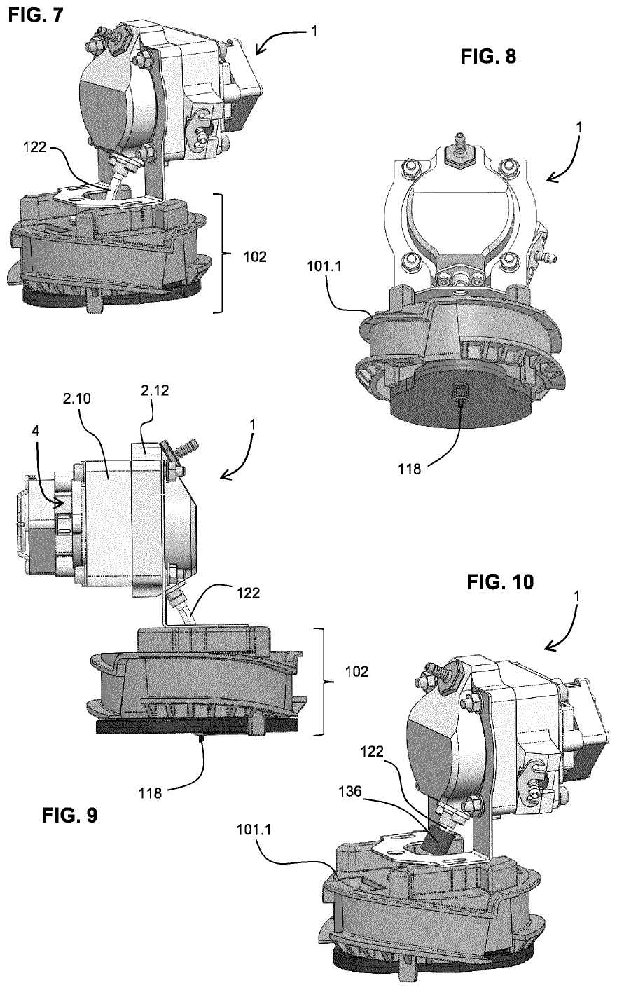

The sanitization unit 1 may be located above at least one of the beverage preparation unit 102 and the liquid supply unit 104. The sanitization unit 1 may be located in an uppermost position of the beverage preparation machine 101, for example at the highest position, for example on a head 101.1, of the beverage preparation machine 101 as illustrated in FIGS. 7 to 10. As the sanitization unit 1 lies above the beverage preparation unit 102, the gravity can enhance the evacuation of the liquid out of the irradiation chamber 2 via the liquid inlet 10 and the drainage of a discharge line 122 toward the beverage preparation unit 102 and from there to the product container.

The liquid supply unit 103 may comprise at least one supply line, which is configured to guide the liquid, and a liquid pump 112, which is configured to displace the liquid in this supply line. The beverage preparation unit 102 may be configured to receive a product container. The liquid pump 112 may be configured to dispense liquid to the beverage preparation unit 102 under a pressure higher than 5 bar in order to achieve a high velocity, in particular in the product container, so as to properly dissolve or extract the nutritional elements. The liquid pump 112 may for example be a type EK2 piston pump.

The beverage preparation machine 101 may further comprise an air supply 108, which is fluidly connected to the air inlet 14 so as to let air enter the irradiation chamber 2 via the air inlet 14 and evacuate the liquid out of the irradiation chamber 2 via the liquid outlet 12. The air supply 108 may comprise at least one fluid line to guide air and an air pump 116 configured to move the air in this fluid line. The air entering the air inlet 14 may be compressed air, for example under an air pressure of between 0.5 to 2.0 bar.

The beverage preparation unit 102 may comprise an opening device for opening the product container, for example a hollow needle 118, as illustrated in FIGS. 4 to 6, 8 and 9. The hollow needle 118 may be configured for piercing a lid of the product container and for injecting the liquid therein. The beverage preparation unit 102 may further comprise a not shown opening actuator configured for actuating the opening device so as to open the product container.

The beverage preparation machine 101 may further comprise an upstream duct 120 and a discharge line 122, which are arranged respectively upstream and downstream of the sanitization unit 1. The upstream duct 120 may be fluidly connected to the liquid inlet 10, and a discharge line 122 may be fluidly connected to the liquid outlet 12.

The upstream duct 120 may be arranged to guide the liquid supplied by the liquid supply unit 103 to the sanitization unit 1. The liquid supply unit 103 may comprise a liquid tank 104, for example a water tank, and the liquid may be tapwater. The discharge line 122 may be arranged to guide the liquid between the sanitization unit 1 and the beverage preparation unit 102 and from there to the not shown product container via the hollow needle 118.

As illustrated in FIGS. 4 to 6, the beverage preparation machine 101 may further comprise:
a heating system 130 arranged between the liquid supply unit 103 and the sanitization unit 1, the heating system 130 being configured to heat up the liquid during preparation of a beverage,
a machine control unit 132 configured to control at least one of: the sanitization unit 1, hence the irradiation device 4, the liquid pump 112, the air pump 116, the heating system 130 and the non-return valve 20, and
a flowmeter 134 arranged between the liquid tank 104 and the liquid pump 112 to measure the flow rate of liquid.

The heating system 130 and the flowmeter 134 are fluidly connected to the supply line that fluidly connects the liquid supply unit 103 and the liquid inlet 10. The machine control unit 132 may be configured to control the heating system 130, the flowmeter 134, the liquid pump 112 and the air pump 116.

As illustrated in FIG. 10 the beverage preparation machine 101 may further comprise a heating device 136, which is arranged to transfer heat to at least a portion of the discharge line 122, for example substantially to the whole of the discharge line 122. The discharge line 122 may be made of a thermally conductive material, for example of metal, in particular of stainless steel. The heating device 136 is not shown on the partial FIGS. 7, 8 and 9.

The heating device 136 may comprise a heating element, a temperature sensor and a heat control unit. The heating element may be arranged around the discharge line 122 and have the shape of a cartridge heater. The temperature sensor may be arranged to measure the temperature of the heating element or of a portion of the discharge line 122. The temperature sensor may be a thermal measuring resistance of the NTC type. The heat control unit may be configured to control the temperature of the heating element so as to heat the liquid present in the discharge line 122 up to a temperature ranging from 65° C. to 90° C., for example about 75 degrees.

The beverage preparation machine 101 may further comprise an end valve 140, which is arranged in a line fluidly connecting the liquid outlet 12 and the beverage preparation unit 102. The end valve 140 may be configured to open only in one direction from the liquid outlet 12 to the beverage preparation unit 102 and only when the pressure difference across the end valve 140 exceeds a given pressure difference threshold, for example of about 4 bar. In the example of FIGS. 4 to 6, the end valve 140 may be a check valve or non-return valve and it may be located immediately upstream the opening device (hollow needle 118).

When the beverage preparation machine 101 is in service and preparing a beverage, the flow restrictor 12.1 may make the liquid follow a long swirly path inside the irradiation chamber 2. Thus, the liquid may be irradiated long enough to be properly sanitizated. Then, the flow restrictor 12.1 may allow for a high liquid flow rate, such that the sanitized liquid gets injected into the beverage preparation unit 102 and then into the product container to prepare the beverage.

When the beverage preparation machine 101 is in service, it may carry out a beverage preparation method according to an embodiment for preparing a beverage by delivering liquid to the beverage preparation unit 102. The beverage preparation method comprises:
activating the liquid supply unit 103 to deliver a liquid via the liquid inlet 10 to the irradiation chamber 2, wherein the flow restrictor 12.1 restricts the liquid flow via the liquid outlet 12 to the restricted flow rate when the pressure difference across the flow restrictor 12.1 is below the predetermined threshold value, and
activating the irradiation device 4 at least during the delivery of liquid to the irradiation chamber 102 in order to sanitize the delivered liquid, and
keeping the liquid supply unit 103 running, wherein the flow restrictor 12.1 allows for a liquid flow, via the liquid outlet (12) and to the beverage preparation unit (102), with the discharged flow rate when the pressure difference across the flow restrictor 12.1 is equal to or greater than the predetermined threshold value,
preferably, increasing the liquid flow supplied to the irradiation chamber 2 via the liquid inlet 10 after the pressure difference across the flow restrictor 12.1 has become equal to or greater than the predetermined threshold value,
optionally, regulating the liquid flow supplied to the irradiation chamber 2 via the liquid inlet 10 so as to allow for the liquid to remain in the irradiation chamber 2 for a predefined residence time before flowing out of the irradiation chamber 2 via the liquid outlet 12, and
preparing a beverage in the beverage preparation unit 102 with the sanitized liquid, for example by injecting the sanitized liquid to the beverage preparation unit 102.

Then, the liquid may be discharged to flow into the beverage preparation unit 102, so as to be injected at a sufficient velocity into the beverage preparation unit 102 and possibly into the product container in order to prepare the beverage.

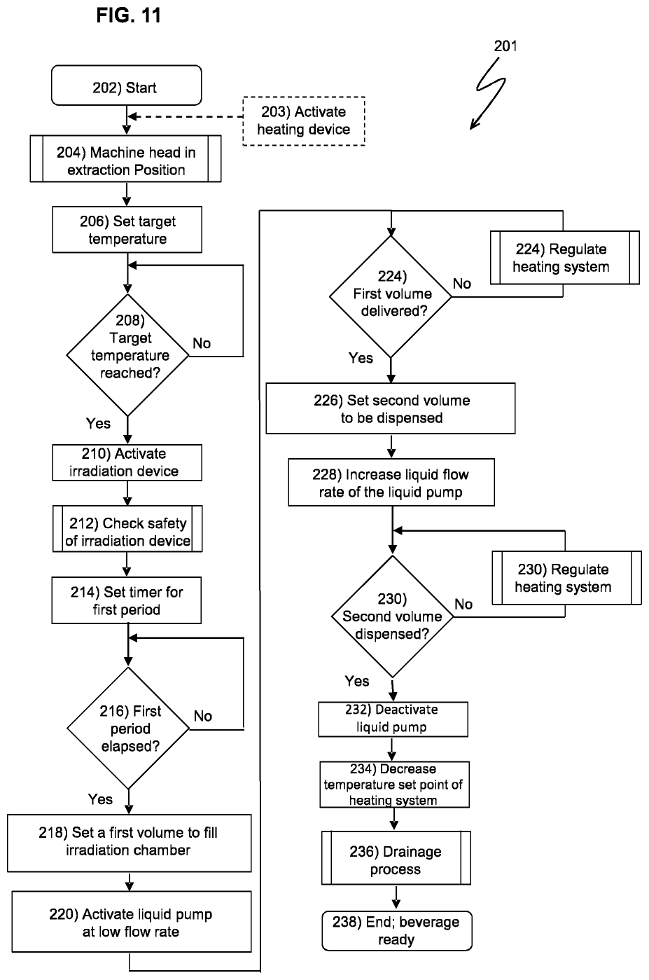

FIG. 11 illustrates some steps of a beverage preparation method 201 when the beverage preparation machine 101 and the sanitizing unit 1 are in service. The indications written in FIG. 11 are merely added to enhance the legibility of the flowchart. Further steps may be performed that are not illustrated in FIG. 11.

The beverage preparation method 201 may be controlled by the machine control unit 118. The irradiation chamber 2 is empty at the beginning of the beverage preparation method 201. The beverage preparation method 201 may comprise:
202) Starting the beverage preparation method 201, hence also the beverage preparation machine 101.
203) The heating device 136 may be activated during a brief disinfection period, in order to disinfect the discharge line 122, for example by heating up the discharge line 122 at about 75° C.
204) Placing the machine head 101.1 in an extraction position.
206) Setting a target temperature of the heating system 130, e.g. 30 to 43° C. in the case of an infant formula preparation.
208) Checking whether the set target temperature has been reached. If not, waiting until the set target temperature has been reached.
210) Activating the irradiation device 4, for example powering on the UV LEDs 6, during a first period, e.g. of 5 s, before filling the irradiation chamber 2 with the liquid.

The irradiation device 4 may also be activated at the start of a beverage preparation, preferably at least 10 seconds before the liquid pump 112 is activated, and then remain activated up to the end of the beverage preparation including a drainage process as described hereinbelow.
212) Checking safety of the irradiation device 4, for example of the UV LEDs 6.
214) Setting a timer for the first period during which the irradiating device 4 remains activated.
216) Checking whether or waiting until the first period is ended, in which case the set target temperature has been reached.
218) Set a first volume for the liquid to be introduced into the irradiation chamber 2. The first volume may be set substantially equal, or strictly equal, to the volume of the irradiation chamber 2.
220) Activating the liquid pump 112 to pump liquid, e.g. water, from the liquid supply 104 and to push the liquid through the heating system 130 and the upstream duct 120 in order to fill the irradiation chamber 2.

The liquid is introduced into the irradiation chamber 2 through the liquid inlet 10, for example along the injection direction D10 that is tangential to the upper surface 4.1 of the irradiation device 4. The liquid may be introduced at a low flow rate, for example ranging from 50 to 200 mL/min) in order to avoid or minimize the formation of air bubbles by cavitation, hence to maximize the fluence rate of the UV light in the liquid introduced in the irradiation chamber 2.

During the filling of the irradiation chamber 2, the air held in the irradiation chamber 2 is pushed toward the liquid outlet 12, the beverage preparation unit 102 and the not shown product container. Due in part to the arrangement of the liquid inlet 10, the liquid outlet 12 and the air inlet 14, there remains no air in the irradiation chamber 2, thus avoiding that the liquid flows along too short a path through the irradiation chamber 2.

Optionally, activating the irradiation device 4, for example powering on the UV LEDs 6, during a second period, e.g. of 10 s, upon the start of the beverage preparation in order to ensure that the irradiation chamber 2 is wholly exposed to UV radiations and that the liquid received therein gets sanitized. The irradiation device 4 may then be stopped or deactivated during a heating step performed by the heating element 130. The irradiation device 4 may then be reactivated prior to the activation of the liquid pump 112. In another embodiment, the second period may directly follow the first period, thus involving a continuous activation of the irradiation device 4. These optional steps are not illustrated in the figures.

The irradiation device 4 may be continuously activated (UV LEDs ON) all along the beverage preparation method 201, in order to ensure the sanitization unit 1 gets thoroughly disinfected, even during the injection of air by the air pump 116. Alternatively, the second period and the first period may be separated by a rest period during which the irradiation device is not activated.
224) Checking whether or waiting until the first volume has been delivered by the liquid pump 112, while regulating the temperature of the heating system 130. When performing this step the machine control unit 118 may analyze the data emitted by the flowmeter 134 in order to regulate the liquid flow rate via a control loop controlling the liquid pump 112.

The liquid flow supplied to the irradiation chamber 2 via the liquid inlet 10 may be regulated so as to allow for the liquid to remain in the irradiation chamber 2 for a predefined residence time before flowing out of the irradiation chamber 2 via the liquid outlet 12. Thus, regulating the flow may ensure an appropriate residence time in order to efficiently sanitize the liquid in the irradiation chamber 2.
226) Set a second volume for the liquid to be dispensed in the beverage preparation unit 102 and possibly into a product container. The second volume may be set as the difference between i) the volume required in the beverage recipe and ii) the volume filling the irradiation chamber 2.
228) as of step 220) the irradiation chamber 2 is filled with liquid; activating the liquid pump 112 at an increased flow rate, for example at a maximum admissible flow rate of 400 mL/min, in order to push liquid into the beverage preparation unit 102 and possibly into the product container and hence properly dissolve or extract the product.

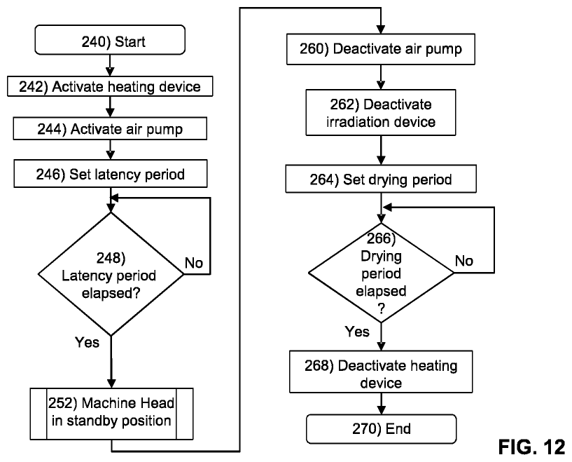

The liquid flows out the liquid outlet 12 on top of the irradiation chamber 2 toward the beverage preparation unit 102 and possibly toward the product container via the discharge line 122 the liquid non-return valve 111 and the opening device (hollow needle 118). The liquid may thus dissolve or extract the product in the product container.
230) Checking whether or waiting until the second volume has been dispensed, while regulating the temperature of the heating system 130.
232) Deactivating the liquid pump 112.
234) Decreasing the temperature set point of the heating system 130, e.g. to a standard preheating temperature.
236) Performing a drainage process (FIG. 12) for draining liquid out of the irradiation chamber 2 and out of the discharge line 122 toward the beverage preparation unit 102 and toward the product container.
238) After the step of dissolving or extracting and after the drainage process, the beverage preparation method 201 is completed and the beverage is ready to drink.

Some steps of the beverage preparation method 201 may be performed in parallel when applicable, as for example steps 212) and 214). Besides, some steps of the beverage preparation method 201 are optional.

FIG. 12 illustrates an embodiment of the drainage process for performing step 236. In order to set the beverage preparation machine 101 ready for preparing the next beverage, the beverage preparation method 201 may further comprise the following drainage process as mentioned in step 236) above:
240) Starting the drainage process.
242) Heating the heating device 136 so as to heat up the discharge line 122 to e.g. about 75° C.
244) Activating the air pump 116 so as to evacuate the liquid out of the irradiation chamber 2, the discharge line 122, the beverage preparation unit 102 and the product container. After a few seconds, e.g. 5 seconds, the irradiation chamber 2, the discharge line 122, the beverage preparation unit 102 and the product container are empty. The period of injection of air may be selected to ensure that the air stream circulates in the whole irradiation chamber 2.

Advantageously, the product container is fully emptied in order to ensure a good nutrition monitoring as performed by a not shown monitoring platform from which the product consumption is directly uploaded by the beverage preparation machine 101.

The entered air delivered by the air pump 116 can also empty the irradiation chamber 2 by pushing the liquid remaining therein through the liquid inlet 10 and the upstream duct 120. Thus, the arrangement of the air inlet 14 in the uppermost region 2.2 facilitates the emptying of the irradiation chamber 2 as well as the emptying of a discharge line 122, of the beverage preparation unit 102 and of the product container.

The flow rate of the entered air may preferably be equal to or lower than the liquid flow rate during the liquid supply to the beverage preparation unit and the product container.
246) Setting a latency of e.g. 3 so as to let the pressure in the beverage preparation machine 101 equilibrate. Meanwhile, the heating device 136 starts drying out the discharge line 120 in order to avoid the growth of microorganisms therein.
248) Checking whether or waiting until the latency has elapsed.
252) Placing the machine head 101.1 in a standby position.
260) Deactivating the air pump 116.
262) Deactivating the irradiation device 4.
264) Setting a timer for a drying period of e.g. 5 minutes in order to dry out the discharge line 122.
266) Checking whether or waiting until the drying period has elapsed.
268) Deactivating the heating device 136.
270) End of drainage process; the beverage preparation machine 101 is ready for preparing the next beverage.

Besides, the beverage preparation method 201 may also include the following step, which is not shown in FIGS. 13 and 14: Regularly rinsing the beverage preparation machine 1 and the sanitization unit with a liquid heated up to a disinfection temperature of e.g. 75° C. by the heating system 130. For example, such a rinsing step may be repeated every 24 hours or after a predetermined number of hours has elapsed since the last beverage preparation method has been completed. Such a rinsing step ensures that no biofilm can form in the beverage preparation machine 201.

The present invention has been described in conjunction with various embodiments and implementations as examples. However, other variations can be understood and effected by those persons skilled in the art and practicing the claimed invention, from the studies of the drawings, this disclosure and the independent claims. In the claims as well as in the description the word “comprising” does not exclude other elements or steps and the indefinite article “a” or “an” does not exclude a plurality. A single element or other unit may fulfill the functions of several entities or items recited in the claims.

1. Sanitization unit, for sanitizing a liquid to be delivered to a beverage preparation unit in order to prepare a beverage, the sanitization unit comprising an irradiation chamber configured to receive an amount of liquid, the irradiation chamber comprising: i) a liquid inlet for introducing the liquid into the irradiation chamber and ii) a liquid outlet for discharging the introduced liquid out of the irradiation chamber, the sanitization unit further comprising an irradiation device configured to emit sanitizing radiations into the irradiation chamber so as to sanitize the liquid in the irradiation chamber, and
the liquid outlet comprises a flow restrictor configured to restrict a liquid flow via the liquid outlet to a restricted flow rate when the pressure difference across the flow restrictor is below a predetermined threshold value, and to allow for a liquid flow via the liquid outlet with a discharged flow rate higher than the restricted flow rate when the pressure difference across the flow restrictor is equal to or greater than the predetermined threshold value.

2. Sanitization unit according to claim 1, wherein the flow restrictor is configured such that the restricted flow rate is in a range from 20% to 80%, of the liquid flow rate via the liquid inlet.

3. Sanitization unit according to claim 1, wherein the flow restrictor is configured such that the discharged flow rate is about 100% of the liquid flow rate via the liquid inlet.

4. Sanitization unit according to claim 1, wherein the flow restrictor has a cross-sectional area ranging from 0.28 mm² to 0.79 mm².

5. Sanitization unit according to claim 1, wherein the flow restrictor comprises a non-return valve.

6. Sanitization unit according to claim 1, wherein the predetermined threshold value ranges from 2 bar to 8 bar.

7. Sanitization unit according to claim 1, wherein the liquid outlet is located in a lowermost region of the irradiation chamber.

8. Sanitization unit according to claim 1, wherein the liquid inlet is located in a lateral side region of the irradiation chamber.

9. Sanitization unit according to claim 1, further comprising an air inlet for letting air enter the irradiation chamber and evacuate the liquid out of the irradiation chamber via the liquid outlet.

10. Sanitization unit according to claim 5, further comprising a non-return valve fluidly connected to the air inlet.

11. Sanitization unit according to claim 1, wherein the irradiation device is positioned with respect to the irradiation chamber so as to be distant from the liquid outlet.

12. Beverage preparation machine comprising:
a sanitization unit, for sanitizing a liquid to be delivered to a beverage preparation unit in order to prepare a beverage, the sanitization unit comprising an irradiation chamber configured to receive an amount of liquid, the irradiation chamber comprising: i) a liquid inlet for introducing the liquid into the irradiation chamber and ii) a liquid outlet for discharging the introduced liquid out of the irradiation chamber, the sanitization unit further comprising an irradiation device configured to emit sanitizing radiations into the irradiation chamber so as to sanitize the liquid in the irradiation chamber,
a beverage preparation unit fluidly connected to the liquid outlet for preparing a beverage with the sanitized liquid,
a liquid supply unit fluidly connected to the liquid inlet and configured to supply a liquid to the beverage preparation unit via the liquid inlet, the irradiation chamber and the liquid outlet, and
a flow restrictor arranged between the irradiation chamber and the beverage preparation unit, the flow restrictor being configured to restrict a liquid flow via the liquid outlet to a restricted flow rate when the pressure difference across the flow restrictor is below a predetermined threshold value, and to allow for a liquid flow via the liquid outlet with a discharged flow rate higher than the restricted flow rate when the pressure difference across the flow restrictor is equal to or greater than the predetermined threshold value.

13. Beverage preparation machine according to claim 12, wherein the sanitization unit is located above at least one of the beverage preparation unit and the liquid supply unit.

14. Beverage preparation machine according to claim 12, further comprising a discharge line fluidly connecting the liquid outlet and the beverage preparation unit, the discharge line being preferably dimensioned in accordance with a first requirement for minimizing microbial growth in the discharge line and a second requirement for minimizing a pressure drop across the discharge line.

15. Beverage preparation machine according to claim 12, further comprising a heating device arranged to transfer heat to at least a portion of a discharge line fluidly connecting the liquid outlet and the beverage preparation unit.

16. Method for preparing a beverage, the method comprising:
implementing a beverage preparation machine comprising a sanitization unit, for sanitizing a liquid to be delivered to a beverage preparation unit in order to prepare a beverage, the sanitization unit comprising an irradiation chamber configured to receive an amount of liquid, the irradiation chamber comprising: i) a liquid inlet for introducing the liquid into the irradiation chamber and ii) a liquid outlet for discharging the introduced liquid out of the irradiation chamber, the sanitization unit further comprising an irradiation device configured to emit sanitizing radiations into the irradiation chamber so as to sanitize the liquid in the irradiation chamber, and the liquid outlet comprises a flow restrictor configured to restrict a liquid flow via the liquid outlet to a restricted flow rate when the pressure difference across the flow restrictor is below a predetermined threshold value, and to allow for a liquid flow via the liquid outlet with a discharged flow rate higher than the restricted flow rate when the pressure difference across the flow restrictor is equal to or greater than the predetermined threshold value,
activating the liquid supply unit to deliver a liquid via the liquid inlet to the irradiation chamber, wherein the flow restrictor restricts the liquid flow via the liquid outlet to the restricted flow rate when the pressure difference across the flow restrictor is below the predetermined threshold value,
activating the irradiation device at least during the delivery of liquid to the irradiation chamber in order to sanitize the delivered liquid,
keeping the liquid supply unit running, wherein the flow restrictor allows for a liquid flow, via the liquid outlet and to the beverage preparation unit, with the discharged flow rate when the pressure difference across the flow restrictor is equal to or greater than the predetermined threshold value, and
preparing a beverage in the beverage preparation unit with the sanitized liquid.
